SEQUENCE LISTING (4 sequences)

SEQ ID NO: 1 - DNA, 19 bp, Artificial Sequence
  MCP-1 Forward Primer
cttcctccaccaccatgca

SEQ ID NO: 2 - DNA, 17 bp, Artificial Sequence
  MCP-1 Reverse Primer
ccagccggcaactgtga

SEQ ID NO: 3 - DNA, 20 bp, Artificial Sequence
  KIM-1 Forward Primer
aggaagacccacggctattt

SEQ ID NO: 4 - DNA, 20 bp, Artificial Sequence
  KIM-1 Reverse Primer
tgtcacagtgccattccagt

(19) United States
(12) Patent Application Publication
(10) Pub. No.: US 2021/0283082 A1
(43) Pub. Date: Sep. 16, 2021

(54) PHARMACEUTICAL COMPOSITION FOR PREVENTION OR TREATMENT OF KIDNEY DAMAGE

(71) Applicants: National University Corporation Kanazawa University, Kanazawa-shi, Ishikawa (JP); Kagami Inc., lbaraki-shi, Osaka (JP)

(72) Inventors: Takashi WADA, Ishikawa (JP); Kengo FURUICHI, Ishikawa (JP); Norihiko SAKAI, Ishikawa (JP); Yasunori IWATA, Ishikawa (JP); Akinori HARA, Ishikawa (JP); Yusuke NAKADE, Ishikawa (JP); Kenji HAMASE, Fukuoka (JP); Masashi MITA, Tokyo (JP)

(73) Assignees: National University Corporation Kanazawa University, Kanazawa-shi, Ishikawa (JP); Kagami Inc., Ibaraki-shi, Osaka (JP)

(21) Appl. No.: 16/972,906

(22) PCT Filed: Jun. 6, 2019
(86) PCT No.: PCT/JP2019/022613

§ 371 Date: Feb. 18, 2021

(30) Foreign Application Priority Data
Jun. 7, 2018 (JP) 2018-109740

(51) Int. Cl. A61K 31/198; A61P 13/12; A61K 9/00

(52) U.S. Cl. CPC A61K 31/198; A61K 9/0019; A61P 13/12

(57) ABSTRACT

The present invention pertains to a pharmaceutical composition for the prevention or treatment of kidney disease, an inflammation inhibitor for the kidneys, an inflammatory cell death inhibitor for the kidneys, or a food for the prevention or improvement of kidney disease, that include D-serine or a derivative thereof. In addition, the present invention pertains to the use of D-serine or a derivative thereof, for the production of a pharmaceutical composition for the prevention or treatment of kidney disease.

### FIELD

The present invention relates to a pharmaceutical composition for prevention or treatment of kidney disorder, and specifically it relates to a suppressor of kidney inflammation, to an inflammatory cell death inhibitor, to foods for prevention or treatment of kidney disorder, and to a kidney protective agent.

### BACKGROUND

The kidneys are organs that filter waste products and excess water in blood and excrete them as urine, and in addition to maintaining body fluid homeostasis they also perform a role in blood pressure, hematopoiesis and bone metabolism by endocrine functions. The kidneys can be damaged and undergo reduced function by causes such as immune system disorders, drugs, hypertension, diabetes, hemorrhage or acute drop in blood pressure, infection, or dehydration accompanying burn. Such conditions are referred to as kidney disease, with diabetic kidney disease being one that is caused by diabetes.

Acute kidney injury (AKI) is a kidney disorder that has a course of several hours to several weeks until onset. Acute kidney injury is a state of sudden reduction in renal function caused by ischemia, drugs or endotoxic shock, which produces increased blood concentrations of urea nitrogen or creatinine as body metabolites and symptoms such as abnormal electrolyte metabolism or acidosis, and it is generally diagnosed by a drastic increase in blood creatinine.

Chronic kidney disease (CKD) is a condition attributed to a variety of different kidney disorders, producing either reduced renal function indicated by glomerular filtration rate, or chronic findings (three months or longer) suggesting persistent kidney disorder. Chronic kidney disease affects 13.3 million people in Japan, representing about 13% of the adult population, and as it increases the risk of end-stage kidney disease (ESKD), it poses a threat to national health. No effective method of treatment exists for chronic kidney disease, and as the disease progresses it leads to reduced renal function and symptoms of uremia, finally requiring renal replacement therapy such as dialysis or kidney transplant, thereby creating a huge burden on the health system and the economy (NPL 1). Because chronic kidney disease has no subjective symptoms until renal function is notably reduced, it is desirable to develop useful biomarkers for early detection and inhibited progression.

Biomarkers for kidney disease include creatinine and urea nitrogen (BUN) which are waste products excreted in urine, as well as NGAL expressed by inflammatory cells, and KIM-1 expressed by damaged proximal tubular epithelial cells, but these have been less than satisfactory as early biomarkers. In recent years it has become possible to measure D-amino acids in blood and urine, indicating a potential for D-amino acids as a kidney disease biomarker (PTL 1).

With kidney disease, changes in D-amino acid levels in blood and urine have been reported to be affected by enterobacteria metabolism. In recent metabolomics research it has been shown that short chain fatty acids derived from enterobacteria affect proliferation of regulatory T cells, and that metabolites play an important role in maintenance of homeostasis and protection of organs. However, there has been no mention of the effects of enterobacterial D-amino acids on disorders or protection of the organs.

### CITATION LIST

### Patent Literature

[PTL 1] International Patent Publication No. WO2013/140785

### Non Patent Literature

[NPL 1] KDIGO 2012 Clinical Practice Guideline for the Evaluation and Management of Chronic Kidney Disease, Kidney International Supplements 1 (2013)
[NPL 2] Alexander W. K., Am J Physiol Renal Physiol 2007, 293, F382-F390
[NPL 3] M. Maekawa et al., Chem. Res. Toxicol. 2005, 18, 1678-1682
[NPL 4] M. Orozco-Ibarra et al., Toxicology 229 (2007) 123-135
[NPL 5] R. E. Williams et al., Toxicology 207 (2005) 179-190
[NPL 6] R. E. Williams, E. A. Lock, Toxicology 201 (2004) 231-238

### SUMMARY

### Technical Problem

It is desirable to develop drugs that treat or prevent kidney disease or have protective effects on the kidneys.

### Solution to Problem

As a result of avid research on D-serine which corresponds to disease-related changes in the bodies of kidney disease patients, and on its physiological action, the present inventors have found that D-serine has a protective effect on the kidneys and has a therapeutic and prophylactic effect against kidney disease, and the present invention has been completed based on this finding.

Specifically, the present invention relates to the following inventions:

[1] A pharmaceutical composition for prevention or treatment of kidney disease, comprising D-serine or its derivative.

[2] The pharmaceutical composition according to [1] above, wherein the pharmaceutical composition for prevention or treatment is used for kidney protection or restoration of renal function.

[3] The pharmaceutical composition according to [1] or [2], wherein the kidney disease includes acute kidney injury and chronic kidney disease.

[4] The pharmaceutical composition according to [1] or [2], wherein the kidney disease is ischemic kidney disorder.

[5] The pharmaceutical composition according to any one of [1] to [4] above, which is to be used in local administration, enteral administration or parenteral administration.

[6] The pharmaceutical composition according to any one of [1] to [5] above, which contains a dose of D-serine that adjusts blood D-serine concentration to 1 nmol/mL to 1 μmol/mL.

[7] The pharmaceutical composition according to any one of [1] to [6] above, wherein the derivative is a compound that is converted to D-serine after being administered.

[8] A suppressor of kidney inflammation, comprising D-serine or its derivative.

[9] A kidney inflammatory cell death inhibitor, comprising D-serine or its derivative.

[10] A prophylactic or ameliorative food for kidney disease, comprising D-serine or its derivative.

[11] The prophylactic or ameliorative food for kidney disease according to [10] above, wherein the derivative is a compound that is converted to D-serine after being administered.

[12] A method for prevention or treatment of kidney disease, which includes administering D-serine or its derivative.

[13] D-serine or its derivative, for use in prevention or treatment of kidney disease.

[14] The use of D-serine or its derivative for production of a pharmaceutical composition for prevention or treatment of kidney disease.

[15] The method, D-serine or its derivative, or use according to any one of [12] to [14] above, which is to be used for kidney protection or for restoration of renal function.

[16] The method, D-serine or its derivative or use according to any one of [12] to [15] above, wherein the kidney disease includes acute kidney injury and chronic kidney disease.

[17] The method, D-serine or its derivative or use according to any one of [12] to [16] above, wherein the kidney disease is ischemia-induced or inflammation-induced kidney disorder.

[18] The method, D-serine or its derivative or use according to any one of [12] to [17] above, which is to be used in local administration, enteral administration or parenteral administration.

[19] The method, D-serine or its derivative or use according to any one of [12] to [18] above, which includes a dose of D-serine that adjusts blood D-serine concentration to 1 nmol/mL to 1 μmol/mL.

[20] The method, D-serine or its derivative or use according to any one of [12] to [19] above, wherein the derivative is a compound that is converted to D-serine after being administered.

### Advantageous Effects of Invention

D-Serine in blood exhibits at least one function or effect from among therapeutic effects on kidney disease such as a kidney protecting effect, anti-inflammatory effect and inflammatory cell death inhibiting effect, and renal function restoration effects.

### BRIEF DESCRIPTION OF DRAWINGS

FIG. 1(A) shows a PAS-stained image of a kidney tissue section at 0, 2 and 10 days after ischemia reperfusion. The staining intensity increased at day 10 in the group given water, with visible accumulation of inflammatory cells such as neutrophils and eosinophils, but in the group given D-serine there was no such change in staining intensity. FIG. 1(B) shows blood D-serine concentration after free provision of 20 mM D-serine water.

FIG. 2 shows grades of (A) necrosis, (B) intraluminal debris and (C) brush border region in a kidney tissue section at 0, 2, 5, 7 and 10 days after ischemia reperfusion. It is seen that the numerical values improved in the group given D-serine, compared to the group given water.

FIG. 3(A) shows the results of F4/80 staining of a kidney tissue section at 0 and 5 days after ischemia reperfusion. The staining intensity increased at day 5 in the group given water, with visible accumulation of inflammatory cells such as macrophages and monocytes, but in the group given D-serine there was little change in staining intensity. FIG. 3(B) is a digitized diagram of the F4/80-stained region.

FIG. 4(A) shows a time schedule for addition of D-serine to tubular epithelial cells (TEC), cultured under hypoxic stress conditions. FIG. 4(B) shows changes in KIM-1 expression in response to D-serine dose.

### DESCRIPTION OF EMBODIMENTS

One aspect of the invention relates to a pharmaceutical composition for prevention or treatment of kidney disorder comprising D-serine or its derivative.

D-Serine is an optical isomer of L-serine, one of the constituent amino acids of proteins. According to the invention, the D-serine may be in the form of any salt such as an acidic salt, basic salt or amphoteric salt, so long as it is a physiologically acceptable salt such as a hydrochloride, sulfuric acid salt, nitric acid salt, sodium salt, potassium salt, calcium salt or ammonium salt.

A D-serine derivative is a compound that causes a change in hydrophobic or electrostatic property, or which allows the D-serine concentration in blood or tissues to be adjusted after being administered. Any D-serine derivative may be used that can adjust the D-serine concentration in blood or tissues to 1 nmol/mL to 1 μmol/mL. Examples of D-serine derivatives include compounds in which the carboxy, amino or hydroxyl groups of D-serine are protected or substituted. The carboxy group may be esterified or amidated. The amino group may be amidated. Hydroxyl groups may be etherified or esterified. Examples of derivatives include D-serine methyl ester and D-serine ethyl ester, and peptides including D-serine, such as dipeptides, tripeptides, oligopeptides or polypeptides.

When a peptide is used, it may consist of serine alone, or it may comprise other amino acids such as alanine, glycine, valine, leucine, isoleucine, threonine, cysteine, methionine, aspartic acid, glutamic acid, asparagine, glutamine, lysine, arginine, phenylalanine, tyrosine, tryptophan or histidine in addition to serine. Amino acids other than D-serine may be in either the L-form or D-form. A D-serine residue, or D-serine produced by decomposition, may also have physiological activity, such as a kidney-protecting effect.

Kidney disease is determined based on proteinuria or glomerular filtration rate (GFR). Chronic kidney disease is diagnosed as such if either or both of the following, i.e.

(1) Clear presence of kidney disorder based on urinalysis, image diagnosis, blood examination or pathology, and particularly proteinuria of 0.15 g/gCr or greater (albuminuria of 30 mg/gCr or greater), and

(2) Glomerular filtration rate of less than 60 mL/min/1.73 m², continues for 3 months or longer. The glomerular filtration rate is determined by calculating the estimated glomerular filtration rate from serum creatinine value, age and gender.

Chronic kidney disease can be caused by a variety of different factors. Risk of kidney disease is associated with diabetes, hypertension, nephritis, polycystic kidney, kidney transplant, dyslipidemia or obesity. According to the invention, therefore, D-serine or its derivative can be administered to a subject having these kidney disease risks. The target of treatment may also be a subject for whom reduced renal function has been assessed based on health examination. An example of health examination is examination of renal function based on urine protein, urine fresh blood, BUN, creatinine or eGFR, and subjects found to have mild abnormality or subjects requiring follow-up in one or more of these examinations may be considered to have risk of kidney disease. The D-serine or its derivative may also be administered to a patient suffering from kidney disease in the hope of restoring or preventing deterioration of renal function.

KIM-1 (Kidney injury molecule-1), used as a biomarker for kidney disease, is a single transmembrane protein with a total length of 104 kDa, whose expression is induced in proximal tubular epithelial cells during repair and regeneration following damage. KIM-1 functions as a receptor for the eat-me signal expressed on the surfaces of cells that have undergone apoptosis, and it is thought that KIM-1 contributes to removal of dead cells.

Acute kidney injury is a disease in which renal function is rapidly reduced during a period of several hours to several days, and it can be mainly classified as ischemic injury or injury due to nephrotoxic substances. When the kidney is not supplied with sufficient blood as a result of shock by hemorrhaging or the like, inflammation occurs in the renal tubules or nephrons of the kidney, leading to loss of function. Nephrotoxic substances include agricultural chemicals, drugs, contrast agents and antibiotics, with renal function being lost due to damage to nephrons by such substances. The kidney disease may be classified as prerenal, renal or postrenal, depending on the site of damage. Prerenal kidney disease, being a systemic disease, is caused by reduced blood flow to the kidneys, and its causes include dehydration, shock, burn, massive hemorrhage, congestive heart failure, hepatic cirrhosis or renal artery stenosis. Renal kidney disease arises from the kidneys themselves, and its causes include blood flow disturbance in the kidneys, glomerular disorder and renal tubular/interstitial disorder. Postrenal kidney disease is due to a problem in the urinary tract below the kidneys. Restoration of renal function can be expected if acute kidney injury is treated early, but if left without proper treatment it can progress to chronic kidney disease. According to the invention, therefore, D-serine or its derivative may be administered for subjects suffering from or at risk of acute kidney injury.

Renal function can be assessed by measuring blood creatinine levels, total blood protein, blood urea nitrogen (BUN) and glomerular filtration rate. Creatinine levels, total blood protein, blood urea nitrogen (BUN) and estimated glomerular filtration rate are commonly measured in health examinations, and the D-serine or its derivative of the invention may be administered to subjects with numerical values below standard levels or having a tendency toward deterioration.

Kidney disease is often accompanied by kidney inflammation, and renal function can be restored by inhibiting such disorder. Kidney disease includes glomerular nephritis and interstitial nephritis, where inflammation takes place in the glomerulus or interstitium. Glomerular nephritis is further classified as acute glomerular nephritis (acute nephritis) or chronic glomerular nephritis (chronic nephritis).

A prophylactic or ameliorative food for kidney disorder is a food indicated for ingestion by a subject suffering from kidney disorder or at risk of suffering from kidney disorder. Such foods include functional foods, health foods and supplements. D-serine or its derivative, or a material comprising it, can be added to any desired food. A food containing D-serine or its derivative or a material comprising it may have any desired dose thereof from the viewpoint of exhibiting a protecting effect or anti-inflammatory effect on the kidneys by the D-serine or its derivative or the material comprising it.

The term “treatment” is considered to be restoration of renal function or alleviation of kidney disorder. Although complete restoration of kidney disorder generally cannot be expected with chronic kidney disease, a composition for treatment according to the invention can be administered for the purpose of inhibiting deterioration. With acute kidney injury, a composition for treatment according to the invention may be administered for the purpose of restoring renal function. According to the invention, “prevention” is inhibiting onset or progression of, or restoration from, kidney disorder, for a subject suffering from or at risk of suffering from impaired renal function.

The D-serine of the invention may be administered by any desired route of administration so long as its concentration is properly adjusted at the site of action. Routes of administration include local administration (percutaneous, inhalation, enema, eye drop, ear drop, pernasal or intravaginal), enteral administration (oral, tubal or enteral), or parenteral administration (intravenous, transarterial, transdermal or intramuscular injection).

D-Serine has been reported to produce nephrotoxicity by intraperitoneal administration in rats (NPL 2: Alexander W. K., Am J Physiol Renal Physiol 2007, 293, F382-F390; NPL 3: M. Maekawa et al., Chem. Res. Toxicol. 2005, 18, 1678-1682; NPL 4: M. Orozco-Ibarra et al., Toxicology 229 (2007) 123-135; NPL 5: R. E. Williams et al., Toxicology 207 (2005) 179-190; NPL 6: R. E. Williams, E. A. Lock, Toxicology 201 (2004) 231-238). In these publications it is disclosed that administration in doses exceeding 250 mg/kg, such as 800 mg/kg (NPL 3), 400 mg/kg (NPL 4) or 250 mg/kg (NPLs 5 and 6) causes nephrotoxicity due to oxidative stimulation or DAO action. Specifically, it is thought that D-serine is metabolized by the action of D-amino acid oxidase (DAO) in the proximal tubules, and that the active oxygen species generated by this reaction causes cell damage and leads to necrosis.

In the research in rats in NPL 2, the renal effects of administering D-serine at 0.25, 0.76, 2.54 and 7.6 mmol/kg by intraperitoneal administration are examined. Since the molecular weight of D-serine is 105, these doses correspond to 26 mg/kg, 80 mg/kg, 267 mg/kg and 800 mg/kg, respectively. When D-serine was intraperitoneally administered at such concentrations, there was no effect on amino acid excretion and no toxicity was exhibited after 2 hours with 26 mg/kg or 80 mg/kg, but with 267 mg/kg and 800 mg/kg the amino acid excretion significantly increased and toxicity of D-serine on the kidneys was exhibited. When amino acid excretion after 4 hours was examined, no toxicity was exhibited with 26 mg/kg but very weak toxicity was exhibited with 80 mg/kg. Therefore, the non-toxic level (NOAEL) in rats may be determined to be 25, 30, 40 or 50 mg/kg. Thus, setting the dose of D-serine to the NOAEL, and also setting a safety factor of 10 in consideration of the difference in species, 2.5 to 5.0 mg/kg/day may be set as the upper limit for dosage. A different safety factor may be optionally set. The upper limit for dosage is preferably 5.0 mg/kg/day, more preferably 4.0 mg/kg/day, even more preferably 3.0 mg/kg/day and most preferably 2.5 mg/kg.

In the Examples described below, mice were freely provided with drinking water containing 20 mM D-serine until the blood D-serine concentrations reached 100 nmol/mL, and at this concentration D-serine was able to exhibit a protecting effect on the kidneys. Without being limited to any particular theory, the effects of the invention, i.e. a therapeutic effect and protective effect on kidney disorder, a renal function restoration effect, an effect of suppressing inflammation of the kidneys and an effect of inhibiting inflammatory cell death, can be exhibited by a D-serine blood concentration of 1 nmol/mL to 1 μmol/mL, although it is necessary to also consider the difference in species. The D-serine blood concentration is preferably 5 nmol/mL or greater, more preferably 10 nmol/mL or greater and even more preferably 50 nmol/mL or greater. The D-serine blood concentration is also preferably 1 μmol/mL or lower, more preferably 0.5 μmol/mL or lower and even more preferably 0.1 μmol/mL or lower.

The pharmaceutical composition of the invention may also include pharmacologically acceptable carriers, diluents or excipients in addition to the D-serine or its derivative. The pharmaceutical composition of the invention may also comprise an anti-inflammatory drug or renal function-improving agent in addition to the D-serine or its derivative. Such a pharmaceutical composition may be provided in a form suited for local administration (skin, inhalation, enema, eye drop, ear drop, nasal or intravaginal), enteral administration (oral, tubal or enteral) or parenteral administration (intravenous, transarterial, transdermal or intramuscular injection), with no limitation to these routes of administration.

The therapeutic agent or pharmaceutical composition of the invention may be formulated in a dosage form selected to be suitable for the route of administration. The dosage form may be designed as a tablet, capsule, liquid drug, powdered drug, granules or a chewable agent for use in oral administration, or as an injection, powdered drug or infusion preparation for parenteral administration. These formulations may also include various types of adjuvants such as carriers or other auxiliary agents that are used in drugs, including stabilizers, antiseptic agents, soothing agents, flavorings, taste correctives, aromatics, emulsifiers, fillers and pH adjustors, in ranges that do not interfere with the effect of the composition of the invention.

All of the publications mentioned throughout the present specification are incorporated herein in their entirety by reference. The examples of the invention described below are intended to serve merely as illustration and do not limit the technical scope of the invention. The technical scope of the invention is limited solely by the description in the Claims. Modifications of the invention, such as additions, deletions or substitutions to the constituent features of the invention, are possible so long as the gist of the invention is maintained.

### EXAMPLES

### Example 1: Inducing Kidney Disorder by Ischemia Reperfusion

1. Materials and Method

(1) Research Ethics

All of the experiments were conducted according to facility guidelines, under the approval of the Animal Experiment Committee of the facility.

(2) Materials

Amino acid enantiomers and HPLC-grade acetonitrile were purchased from Nacalai Tesque, Inc. (Kyoto). HPLC-grade methanol, trifluoroacetic acid and boric acid were purchased from Wako Pure Chemical Industries, Ltd. (Osaka). The water was purified using a Milli-Q gradient A10 system.

(3) Animals

The animals were reared with free access to water and feed under conditions with an SPF environment and 12-hour light-dark cycling. C57BL/6J mice were purchased from Clea Japan, Inc. (Osaka).

(4) Renal Ischemia Reperfusion Treatment

Male 12- to 16-week-old mice were treated by renal ischemia reperfusion (hereunder also referred to as “I/R”). The renal pedicles were clamped with a non-traumatic clip (Natsume Seisakusho, Tokyo) under pentobarbital anesthesia to elicit ischemia. The clips were released after 40 minutes. The body temperature was kept at 37° C. during treatment.

The mice were reared with free access to water (control) or 20 mM D-serine-containing water, prior to 14 days after IR treatment. On day 0, 2, 5, 7 and 10 after IR treatment, the kidney tissues were sampled.

(5) D-Serine Blood Concentration

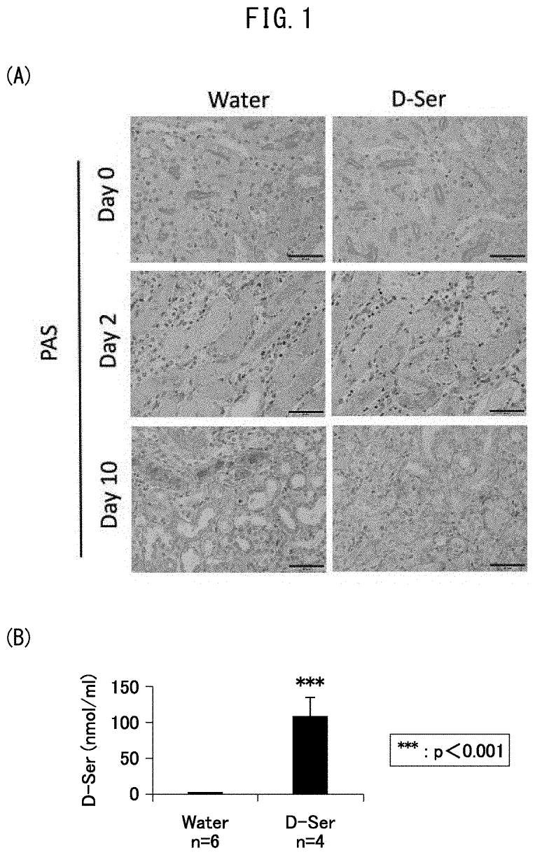

The mice were reared in an environment allowing free access to water (control) or 20 mM D-serine-containing water, and the blood D-serine concentrations were measured. The blood D-serine concentrations were determined by total amino acid optical isomer analysis using a D, L-amino acid simultaneous high sensitivity analysis system developed by Zaitsu et al. (Japanese Patent No. 4291628). The details regarding the analysis conditions for each amino acid are described in Miyoshi Y. et al., J. Chromatogr. B, 879:3184(2011) and Sasabe, J. et al., Proc. Natl. Acad. Sci. U.S.A., 109:627(2012). In brief, serum and urine amino acids were fluorescently derivatized with NBD-F (4-fluoro-7-nitro-2,1,3-benzooxadiazole, Tokyo Kasei Kogyo Co., Ltd.) and supplied to an HPLC system (NANOSPACE SI-2 Series, product of Shiseido Corp.). NBD-amino acids separated two-dimensionally using an in-house reversed-phase column with an octadecyl group as the stationary phase and a chiral column with an amino acid derivative as the stationary phase, were quantitatively analyzed after detection at an excitation wavelength of 470 nm and a detection wavelength of 530 nm. In the mice reared with 20 mM D-serine-containing water, the blood D-serine concentration reached 100 nmol/mL (FIG. 1B).

Staining

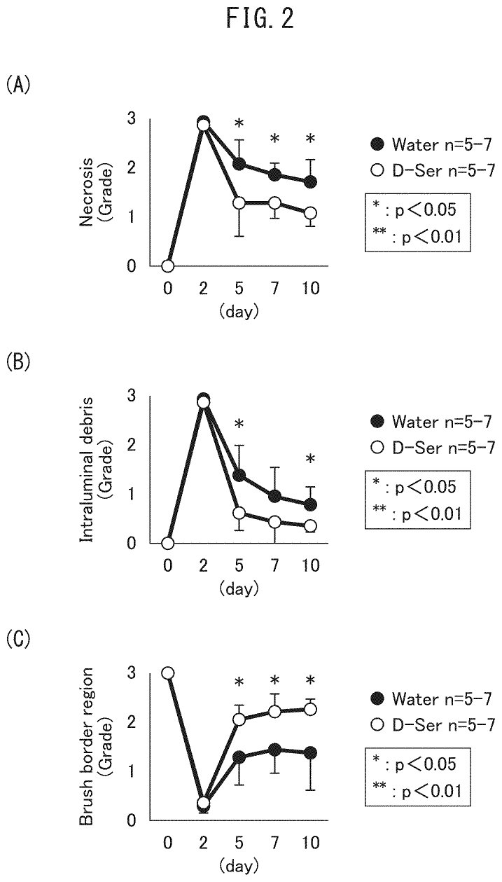

Kidneys sampled on day 0, 2 and 10 were fixed with 10% neutral buffered formalin and embedded in paraffin, and then stained with periodic acid-Schiff (PAS staining). The stained slices were photographed with a bright-field microscope (FIG. 1). Debris stained with PAS (skin boundary) or brush border (skin boundary and cortical region) were quantitatively evaluated in at least 10 different locations. Renal tubule necrosis (A), intraluminal debris (B) and brush border region (C) were evaluated by ATN score (0: none, 1: mild, 2: moderate, 3: severe), with the results shown in FIGS. 2(A) to (C). Sample evaluation was carried out blind.

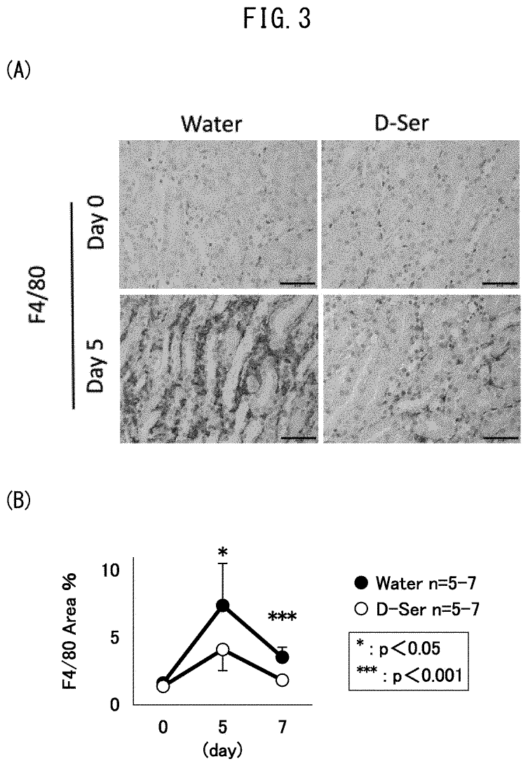

Renal samples harvested on day 0 and day 5 were fixed with 10% neutral buffered formalin and supplied for immunostaining using F4/80 antibody (Invitrogen, catalog #: MF48,000). F4/80 is a protein expressed specifically by macrophages, and it allows visualization of macrophages in kidney tissue. In the water-administered control group, cells stained with F4/80 were found accumulated in kidney tissue (day 5), but in the D-serine-administered group such cellular accumulation was minimal (FIG. 3(A)). The regions stained with F4/80 in the kidney tissue harvested on day 0, 5 and 7 were measured and shown (FIG. 3(B)).

### Example 2: Hypoxic Stress on Tubular Epithelial Cells (TEC)

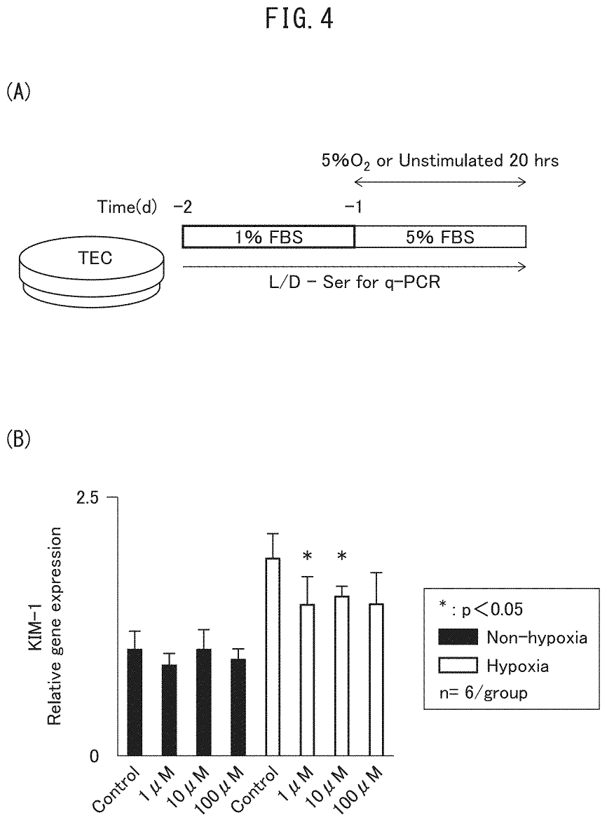

A mouse tubular epithelial cell line, mProx24cell, was provided from Sugaya (St. Marianna University School of Medicine, Tokyo). The cells were cultured in DMEM culture medium containing 5% fetal bovine serum (FBS) and 1% penicillin and streptomycin. The cultured cells were seeded using 1% FBS-added DMEM medium at 1.0×10⁶ cell/well, and cultured for 24 hours in a 37° C., 5% CO₂ and 20% O₂ humidified atmosphere. In the hypoxic stress group, the 24 hours of culturing was followed by an additional 20 hours of culturing in 5% FBS-added DMEM medium in a 5% CO₂ and 5% O₂ humidified atmosphere, while in the non-hypoxic-stress group, it was followed by an additional 20 hours of culturing in a 5% FBS-added DMEM medium in a 5% CO₂ and 20% O₂ humidified atmosphere (FIG. 4(A)). D-Serine was added as a test drug at 1 μM, 10 μM and 100 μM to each DMEM medium, but with no D-serine added to the control.

Measurement of KIM-1 Gene Expression

The cultured cells were collected and the total RNA was extracted using a High Pure RNA Isolation Kit (Roche Diagnostics, Tokyo). Quantitative real-time PCR using SYBR Green fluorescence (Bio-Rad, Tokyo) was conducted with a Villa 7 Real-Time PCR System (Thermo Fisher Scientific, Tokyo), using the following primers. The data was analyzed by the delta-delta Ct method (FIG. 4(B)).

[Sequence Listing]
(SEQ ID NO: 1)
MCP-1 Forward: 5′-cttcctccaccaccatgca-3′
(SEQ ID NO: 2)
MCP-1 Reverse: 5′-ccagccggcaactgtga-3′
(SEQ ID NO: 3)
KIM-1 Forward: 5′-aggaagacccacggctattt-3′
(SEQ ID NO: 4)
KIM-1 Reverse: 5′-tgtcacagtgccattccagt-3′

1. A method for prevention or treatment of kidney disease, comprising:
administering D-serine or its derivative to a subject in need thereof.

2. The method according to claim 1, wherein the D-serine or its derivative is used for kidney protection or restoration of kidney function.

3. The method according to claim 1, wherein the kidney disease includes is acute kidney injury and or chronic kidney disease.

4. The method according to claim 1, wherein the kidney disease is ischemic kidney disorder.

5. The method according to claim 1, wherein the administration is local administration, enteral administration or parenteral administration.

6. The method according to claim 1, wherein a dose of the D-serine that adjusts blood D-serine concentration to 1 nmol/mL to 1 μmol/mL, is administered.

7. The method according to claim 1, wherein the derivative is a compound that is converted to D-serine after being administered.

8. A method for suppressing kidney inflammation, comprising administering D-serine or its derivative to a subject in need thereof.

9. A method for inhibiting kidney inflammatory cell death, comprising administering D-serine or its derivative to a subject in need thereof.

10. (canceled)

11. The method according to claim 1, wherein the derivative is a compound that is converted to D-serine after being administered.

12.-14. (canceled)

15. The method according to claim 1, wherein the kidney disease is ischemia-induced or inflammation-induced kidney disorder.

16.-18. (canceled)
